(19) United States
(12) Patent Application Publication
(10) Pub. No.: US 2023/0024425 A1
(43) Pub. Date: Jan. 26, 2023

(54) ARTERIAL STENOSIS DETECTION AND QUANTIFICATION OF STENOSIS SEVERITY

(71) Applicants: Amir Landesberg, Haifa (IL); Amit Livneh, Hofit (IL); Tomer Heitner, Haifa (IL)

(72) Inventors: Amir Landesberg, Haifa (IL); Amit Livneh, Hofit (IL); Tomer Heitner, Haifa (IL)

(21) Appl. No.: 17/783,763

(22) PCT Filed: Dec. 9, 2020
(86) PCT No.: PCT/IB2020/061693

§ 371 Date: Jun. 9, 2022

Related U.S. Application Data
(60) Provisional application No. 62/945,305, filed on Dec. 9, 2019.

(51) Int. Cl. A61B 5/02; A61B 5/0295; A61B 5/00; A61B 5/0285; A61B 5/352; A61B 5/021

(52) U.S. Cl. CPC A61B 5/02007; A61B 5/0295; A61B 5/7239; A61B 5/0285; A61B 5/6828; A61B 5/4842; A61B 5/352; A61B 5/02125

(57) ABSTRACT

A method measures a perfusion wave upstroke associated with leg perfusion dynamics, the perfusion wave upstroke including two phases, an initial slow phase and a fast-rising phase, and using prolongation of the slow phase to detect a presence of arterial stenosis and to assess stenosis severity.

### FIELD OF THE INVENTION

The present invention relates generally to arterial stenosis detection and quantification of stenosis severity, such as the quantification of peripheral tissue ‘perfusion deficit’ and measurements of indices that characterize the changes in the microcirculation impedance as prolongation of the maximum acceleration time and the slow phase of the perfusion wave upstroke and the filling rate to arterial tree, to provide sensitive indices for arterial stenosis detection.

### BACKGROUND OF THE INVENTION

Peripheral arterial disease (PAD) is a pandemic affecting more than 200 million patients^{1,2}. PAD is one of the most common first presentations of cardiovascular disease³. The hazard ratio of diabetics having PAD is 2.98, twice the hazard ratio of other cardiovascular diseases³.

The ankle-brachial index (ABI) is a simple method for detecting PAD with a well-established prognostic value⁴. A meta-analysis of 16 studies has revealed that low ABI (<0.9) carries a hazard ratio of 4.2 in men and 3.5 in women for cardiovascular death within ten years⁴. Moreover, the severity of coronary disease is significantly higher in patients with low ABI⁵. Consequently, the guidelines have recommended to screen the population at risk by measuring the ABI⁶. Intriguingly, 55% of the patient with PAD based on ABI<0.9 are asymptomatic⁷.

A review of 8 studies examined the detection sensitivity of low ABI (<0.9) in identifying significant arterial stenosis (>50%) in comparison with standard imaging such as Doppler ultrasonography, digital subtraction angiography, and magnetic resonance angiography⁸. The review has reported that the sensitivity of the ABI is low, from 15% to 79%, especially in the elderly and diabetics⁸. A study that compared the ABI against angiography in patients that incidentally underwent angiography has found that the sensitivity of ABI is 69.3%⁹. A Swedish group has investigated the sensitivity of low ABI (ABI<0.9) in asymptomatic subjects, from a random sample of subjects aged 70-72, by comparing it to whole-body magnetic resonance angiography¹⁰. They have reported that the sensitivity of low ABI in detecting significant stenosis (>50% narrowing) was only 20% on the right leg and 15% on the left leg¹⁰.

### SUMMARY OF THE INVENTION

The available noninvasive methods for assessing the peripheral perfusion are: ankle-brachial index (ABI), impedance plethysmography (IPG), photoplethysmography (PPG), duplex/doppler ultrasound, computer tomography angiography (CTA) and magnetic resonance angiography (MRA).

The limitations of the known solutions are:

1. The ABI suffers from low sensitivity and inaccuracy (as discussed below)

2. Although IPG was used in the past, devices that have used the IPG failed in the signal processing and the identification of the various phased of the perfusion wave. The old devices were also cumbersome and expensive. Thus, they did not realize the advantage over the other technologies and did not extract the features that are presented in the present disclosure.

3. Photoplethysmography (PPG) is impractical in monitoring the lower limbs, especially in the elderly and diabetics. In these patients the dermis is thick and there is a decrease in the underlying microvasculature. Moreover, has is described here, even when the same precise algorithm is applied to the IPG and PPG, the feature extraction form the PPG is significantly less precise than from the IPG. The PPG signal is very weak and noisy. Furthermore, The IPG measure the changes in the entire cross-section of the measured region that includes also the major large arteries and veins, while the PPG measures the changes in the close vicinity of the transudes, since the tissue absorbs the optical signal. Therefore, the PPG measures the changes in the microcirculation. The ability to measure the dynamic of the large arteries is additional advantages of the IPG over the PPG.

4. The duplex/doppler ultrasound is cumbersome and requires trained medical staff. Moreover, it can be used only for performing sporadic follow-up measurements.

5. CTA—is expensive, utilizes X-ray radiation and iodine dye. The X-ray increases the risk of cancer and utilization of iodine dye carries some risk to the kidneys.

6. MRI—is cumbersome, slow and expensive, and cannot be used for frequent monitoring.

The present invention presents novel methods and peripheral perfusion dynamics indices that may outperform the ABI or the PPG (as described below) and highlights the advantages of quantifying the perfusion dynamics in detecting arterial stenosis. Confirming this result in future studies may significantly improve the screening of patients with hypertension, decision making and early detection of restenosis following revascularization.

Some of the novel features of the invention include, without limitation:

The perfusion wave upstroke in the arterial tree below the stenosis consists of two phases, an initial slow-phase and a fast-rising phase.

The slow phase prolongation may detect the presence of arterial stenosis and assess its severity with higher sensitivity than the ABI.

The perfusion waves at the proximal arterial tree and the microcirculation have different phase delay that depends on the severity of arterial stenosis. Monitoring the perfusion (volume changes) at the microcirculation and the arterial level can provide accurate assessment of the “perfusion deficit”. Larger perfusion deficit is associated with large vasodilation, greater initial flow to the microcirculation and longer initial phase with sallower changes in the arterial volume (diameter) and pressure. Wavelet coherence analysis of both legs further improves the detection sensitivity of arterial stenosis.

The method of the invention overcomes the limitations of the ABI:
It improves the sensitivity of arterial stenosis detection by quantifying the changes in the above described dynamics, while the ABI is based on a single point in time—the point of peak systolic pressure (discussion below).
It enables to differentiate between general arteriosclerosis (stiffening of the arterial wall) and development of focal stenosis (discussion below). Atherosclerosis causes two main different problems: (a) general arteriosclerosis, i.e. arterial stiffening (which decreases the arterial compliance and increases the pulse wave velocity) and (b) focal arterial stenoses that limit the blood flow. The latter requires interventions and revascularization. The presence of arteriosclerosis may mask the existence of stenosis, and the ABI does not differentiate between the two. It is therefore important to detect peripheral artery stenosis in the presence of various degree of arteriosclerosis. The novel indices of the perfusion dynamic may detect stenosis irrespectively of the degree of arteriosclerosis, and overcome a major limitation of the ABI.

The disclosure highlights the feasibility and advantages of quantifying the changes in the pattern of the perfusion dynamics in the presence of arterial stenosis and the strength of and ensuing novel indices. The disclosure reveals that the perfusion wave upstroke can be detected non-invasively by, for example, impedance plethysmography, arterial tonometry, or ultrasound that measure the profile of the arterial flow and invasively by impedance catheter (that measures changes in the arterial diameter) or invasive pressure measurement that provides the entire pressure waveform and not just the peak. The perfusion wave upstroke consists of two distinct phases: an initial slow-phase, and a fast-phase. These differentiation and characterization of the initial phases of the perfusion upstroke was not suggested or described before. The maximum acceleration time (MAT) denoted the beginning of the steep rise of the second phase. Focal stenosis prolongs the peak-perfusion time and the maximum acceleration time (MAT) without affecting the pulse-transit time (PTT) and with minimal effect on the ankle-brachial index (ABI) even in the presence of significant stenosis (discussion below).

In one embodiment, the method measures the perfusion dynamics of the arterial, venous and tissue compartments and compares the changes in the phases in the perfusion of these compartments relative to each other. One noninvasive mode to perform this task is to measure the impedance plethysmography in two arrangements and different frequencies: The main two arrangements are: (1) a longitudinal arrangement (FIGS. 7A and 8a) where the electrodes are aligned one below the other along the leg long axis, and (2) a transversal arrangement, where the electrode are aligned around the circumference of the leg as depicted in FIGS. 7B and 8B.

In the longitudinal arrangement the main arteries and veins are aligned in parallel to the large tissue mass, as depicted in FIG. 7A. Therefore, the total impedance in the longitudinal arrangement is the impedance of three parallel compartments: arterial venous and tissue, as depicted in FIG. 8A. The impedance of the arteries and veins is approximately pure resistance, while the tissue has large capacitive property. The muscles have only small amount of interstitial fluid (about 5% of the tissue mass) that is depicted by the resistor R_{P} (parallel resistance) in FIG. 8A. The cell membranes between the cells provide the capacitive nature of the impedance that is depicted by C_{M} and the resistance within the cell is denoted by R_{S} (serial to the membrane capacitance). At low frequency (well below 100 kHz) the impedance of the muscle mass is huge compare to the low impedance of the arteries and veins. Thus longitudinal impedance plethysmography at low frequency mainly monitors the changes in the arterial and venous systems.

In the transverse cross-section, the large arteries and vein have less significant effects on the transverse impedance since the large vessels run along the long axis and not in the transvers cross-section and they occupy negligible area of the cross-section area, while most of the area contains muscle tissue (FIG. 7B). Moreover, at high frequencies (above 100 kHz) the impedance of the muscle is very low, thus, the transverse impedance plethysmography at high frequency monitor the changes in the tissue impedance, as depicted by FIG. 8B.

Thus, measurement of the tissue impedance, at various arrangement and frequencies, enable to weight the relative perfusion filling of the arteries (early phase) veins (that are slowly modulated by the ventilation) and muscle tissue.

Summary, without limitation, of the novel findings are:
The prolongation of the slow phase duration is proportional to the severity of the stenosis.
The prolonging of the slow phase duration (SPd) upon stenosis development causes the prolongation in the peak-perfusion time, MAT.
There are no significant changes in the second fast upstroke of the perfusion wave, although it is the most prominent part of the perfusion wave. Thus, when the ‘crest time’ is defined by the fast upstroke, the sensitivity of the crest time is reduced.
Quantification of the slow-phase duration and the PTT provide a novel method to differentiate between the arteriosclerosis (shortening of the PPT) and development of stenosis (prolongation of SPd, MAT peak perfusion time). The novel indices can detect stenosis irrespectively of the degree of arteriosclerosis (unlike the ABI).

Moreover, wavelet coherence analysis of the perfusion waves from both legs has demonstrated that arterial stenosis decreases the correlation and creates overt phase delay between the perfusion waves from both legs, at the cardiac frequency band.

The present disclosure suggests that measuring these indices can improve the sensitivity of detecting arterial stenosis and the assessment of the stenosis severity. These measurements may be applied as an alternative or complementary tool for improving diagnosis and follow-up.

The experimental validation study:

Arterial stenosis was emulated by inflating a blood-pressure cuff around the thigh of one leg to 45 and 90 mmHg, in eight healthy volunteers. ABI, impedance plethysmography from both feet and photoplethysmography from the hallux and index finger were continuously measured.

Thigh compression of 45 and 90 mmHg did not significantly affect the ABI and pulse transit time (PTT). However, the perfusion dynamics analysis yielded indices that were proportional to the degree of stenosis. The time to peak perfusion increased from 361±52 ms at baseline to 407±6 and 429±5 ms upon 45 and 90 mmHg compressions (P<0.001). The perfusion upstroke comprised two phases: a slow-phase that was followed by a steep rise. The maximum acceleration time (MAT) denoted the beginning of the steep rise. The MAT increased from 259±3 ms at baseline to 310±6 and 354±5 upon 45 and 90 mmHg compression (P<0.001). The slow-phase duration increased from 73±7 ms at baseline to 117±15 and 157±13 ms upon 45 and 90 mmHg compression (P<0.002). Wavelet coherence analysis revealed a decrease in the correlation and a large phase shift between the legs during thigh compression. The phase difference between the legs increased from 3.3±2.6° at baseline to 34.8±6.6° and 76.3±1.9° under 45 and 90 mmHg compression (p<0.001).

Indices of the perfusion dynamics identify arterial stenosis in the absence of noticeable changes in the ABI and PTT. Prolongation of the slow phase and MAT are sensitive novel indices for detecting stenosis.

### BRIEF DESCRIPTION OF THE DRAWINGS

The present invention will be understood and appreciated more fully from the following detailed description taken in conjunction with the drawings in which:

FIG. 1. The perfusion wave upstroke consists of two phases; an initial slow phase (SF) is followed by a fast phase (FP). The figure presents the impedance plethysmography (IPG), Index finger photoplethysmography (finger PPG) and leg hallux PPG (hallux PPG) during 90 mmHg thigh compression. Four time-points were employed in the analyses: The ECG R wave (dashed line), perfusion wave onset (x), maximum perfusion acceleration point (⋄) and peak perfusion point (o). PTT-Pulse transit time. PPT-Peak perfusion time. MAT- maximum acceleration time. CT-Crest time. PA-Pulse amplitude. SPa-Slow-phase amplitude.

FIG. 2. The longitudinal IPG signals, at low frequency, from the tested legs of two subjects (A,B) revealed conspicuous prolongation of the peak perfusion time (PPT) in the presence of arterial stenosis, mainly due to prolongation of the slow phase duration (SPd). The prolongation of PPT and SPd were proportional to the severity of arterial stenosis (45 versus 90 mmHg compressions).

FIG. 3. The Ankle-Brachial Index (ABI) (A) remained above 0.9 and showed no significant changes between baseline, 45 and 90 mmHg compressions. Conversely, the peak perfusion time (B) the slow phase duration (C) and the maximal acceleration time (D) exhibited significant and proportional prolongation during the emulated arterial stenosis. *p<0.05, **p<0.01, ***p<0.001.

FIG. 4. The wavelet coherence analysis presents conspicuous changes in the correlation (color scale) and phase (arrow angle) between both legs, at the different frequencies (ordinate), during 45 mmHg (A) and 90 mmHg (B) thigh compression. The compressions were imposed from the 4^{th} till the 10^{th} minute (white dashed double-edged arrows). The three frequency bands are denoted as the cardiac (C), Respiratory (R) and Authonmic (A) bands.

FIG. 5. The emulated arterial stenosis decreased the correlation (A) and yielded an overt increase in the phase delay (B) between the tested and the contralateral legs. The correlation was measured at the high (4-6 Hz) cardiac sub-band and the phase shift at the 2-4 Hz sub-band. *p<0.05, **p<0.01, ***P<0.001.

FIGS. 6A and 6B compare the invasive measurement of the fractional flow reserve (FFR) and the noninvasive measurement of the perfusion deficit (PD). FIG. 6A illustrates the currently invasive method to measure the fractional flow reserve (FFR) by catheterization and measurement of the pressures before and after the stenosis. The FFR is the ratio of the pressure after the stenosis to the pressure before it. FIG. 6B illustrates the noninvasive measurement of the slow phase duration (SPd). The term ‘slow phase duration’ and ‘perfusion deficit (PD)’ represent the same measurement. The ‘slow phase duration’ presents what is actually measured while the term ‘perfusion deficit’ represents the meaning of the measurement. The table represents the analogue between the two measurements. A stenosis is denoted as significant stenosis, that requires intervention, when the FFR is smaller than 80% based on the invasive measurement (FIG. 6A) or according to our finding, when the SPd (PD) is larger than 57 msec.

FIGS. 7A and 7B are illustrations of the longitudinal and transverse arrangements of the electrodes of this measurement, respectively.

FIGS. 8A and 8B are circuit diagrams corresponding to the longitudinal and transverse arrangements of the electrodes, respectively.

FIG. 9 is a graphical illustration of a short segment of raw impedance signal (green). The slow swings are attributed to the effect of breathing on the venous return. The cardiac components of the perfusion waves are the fast and smaller complexes, the red circles denote the beginning of a cardiac cycle.

FIG. 10 is a graphical illustration of the raw impedance plethysmographic signal which includes well-defined arterial perfusion waves (e.g. A1, A2) but also significantly distorted perfusion waves (e.g. D1, D2) that are strongly modulated by the respiratory signal.

FIG. 11 is a graphical illustration of the selected stable perfusion complexes (bold lines) that were averaged to define the ‘characteristic perfusion wave’ (red line). The distorted waves (gray lines) were excluded.

FIG. 12 is a graphical illustration of identification of the time points for the segmentation of the perfusion upstroke.

FIG. 13 is a flow chart of the feature extraction algorithm.

### DETAILED DESCRIPTION

The inventors hypothesized that measuring the perfusion rather than the pressure distal to the stenosis, and characterizing the perfusion wave dynamics rather than providing a single value of the peak pressure, would improve the sensitivity of detecting arterial stenosis. The impedance plethysmography (IPG) is affected by the blood volume changes in the arterial, tissue, and venous compartments and comprises precious information about the peripheral perfusion dynamics. Measurements of the IPG at different alignments (longitudinal-to concentrate on the large vessels and transverse-to concentrate on the muscle tissue) and different frequencies (low—to exclude the tissue and high to include the muscle tissue) yield novel indices of the perfusion dynamics that were sensitive to the severity of arterial stenosis.

### Methods

The study for this disclosure was approved by the institutional Helsinki Committee. All subjects gave their written informed consent. Healthy volunteers laid supine and were asked to relax. Femoral artery stenosis was emulated by inflating an adult-sized thigh blood-pressure cuff around the thigh of one leg (tested leg). ECG electrodes were attached to both arms. Impedance electrodes (EL-506, Biopac System Inc. USA) were attached to both feet above and below the ankle (longitudinal setup). The IPG frequency was 100 kHz. A combined pulse oximeter and photoplethysmographic (PPG) sensor (TSD124A, Biopac) was placed on the hallux of the tested leg (hallux PPG). A PPG sensor (TSD200, Biopac) was attached to the index finger of one hand (finger PPG). Simultaneous measurements of the blood pressures (BP A90, Microlife, Taiwan) in the upper arm and the tested leg were used for measuring the ABI. All signals were sampled concurrently and continuously at 1 kHz (Biopac MP 160 system, Biopac).

Two levels of arterial stenosis were emulated by inflating the thigh cuff to 45 and 90 mmHg. Each test included three phases: baseline, compression (arterial stenosis) by cuff inflation, and recovery following cuff deflation. The duration of each compression phase was at least 5 minutes. Subjects were allowed to recover during a 10 minutes rest interval between the two test-sets (45 and 90 mmHg compressions). The ABI was measured at baseline and during the applied cuff compression, after at least 2 minutes of habitation and stabilization.

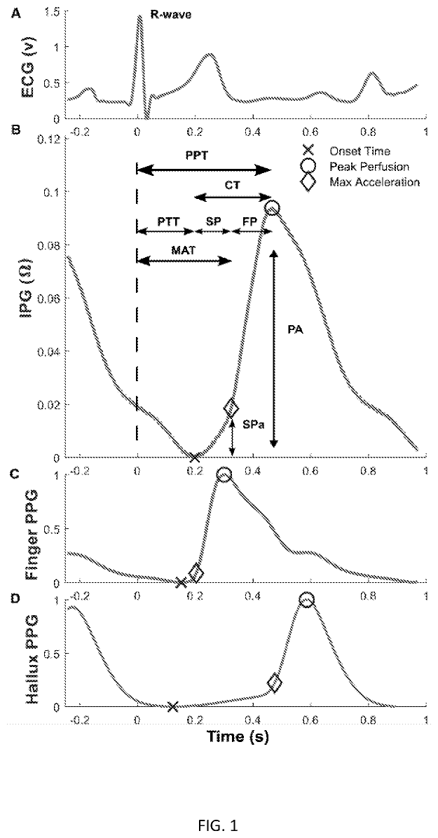

The time-domain analysis employed time-averaged signals. The cardiac waves of the IPG signal are superimposed on slower respiratory waves. To extract the cardiac waves from the IPG signal the signals from each cardiac cycle were aligned relative to the ECG R-wave, and averaged. Three time points were used for segmentation, as shown in FIG. 1:

(1) The perfusion wave onset time (Onset Time), from which the perfusion wave began its rise.

(2) The peak perfusion time (Peak Perfusion), at the peak of the perfusion wave.

(3) The point of maximum acceleration (Max Acceleration), where the second derivative of the signal reached the maximum and the rapid rise began in the wave upstroke.

The time segments were defined as shown in FIG. 1: The pulse transit time (PTT) from the ECG R-wave to the perfusion onset time; The peak perfusion time (PPT) from R-wave to peak perfusion point. The maximum acceleration time (MAT) from the R-wave to the maximum acceleration point. The crest time (CT) from the perfusion onset time to the peak perfusion point, as defined by others^{11,12}. The crest width (CW) as the time period between crossing the 95% threshold of the pulse amplitude (PA), before the perfusion peak and following it, as defined by others¹³. The slow phase duration (SPd) from the onset time to the ‘maximum acceleration point’. The fast phase duration (FPd) from the ‘maximum acceleration point’ to peak perfusion. The crest-time is the sum of the SPd and FPd. The normalized slow phase amplitude (SPa) is the ratio between the perfusion wave rise during the slow phase and the perfusion pulse amplitude.

It is important to note, that the “onset time” is ill-defined in the literature and segmentation of the pulse upstroke was not done before. Consequently, previous measurements failed in the differentiation between arteriosclerosis and stenosis.

Precise identification of the ‘pulse onset time’ and the novel indices as MAT and

SPd requires:

1. Adequate data acquisition, with direct coupling and without high-pass or band-pass filtering, since it will flattened and even eliminate the slow-phase and the definition of the “onset time” will be imprecise.

2. Careful selection of the arterial perfusion complexes and filtration of complexes that are affected by the respiratory wave or autonomic activity or motion artefact. This may be important because the respiratory waves are huge and can be 10 fold larger than the arterial wave. Moreover, the arterial and respiratory waves are very close in their basic frequency.

3. Proper segmentation of the perfusion upstroke, utilizing the above time points and segmentation.

The raw IPG-signal presents the changes in the blood volume in the region of the electrodes. With each heart-beat, blood enters via the arteries and leaves the region through the venous system. The venous drainage is strongly affected by respiration and changes in the thoracic and abdominal pressures. The mean blood volume in the region of interest is constant under stable conditions, but it is modulated by the arterial pulse, the breathing and the slower autonomous activity. The veins are significantly wider than the arteries and contain a significantly larger amount of blood. The venous drainage is affected by breathing, and consequently, the respiratory waves are significantly larger than the arterial perfusion complexes, as depicted in FIG. 9. The arterial perfusion complexes are superimposed on the respiratory waves and are significantly distorted by rapid rises and falls in the respiratory signal.

FIG. 9: A short segment of raw impedance signal (green). The slow swings are attributed to the effect of breathing on the venous return. The cardiac components of the perfusion waves are the fast and smaller complexes, the red circles denote the beginning of a cardiac cycle. The cardiac perfusion waves are severely distorted by the sharp rises and falls of the respiratory signal. For example, the perfusion wave complex at t=402 s was distorted by the sharp rise and the complex at t=404 s was distorted by the sharp decline in the raw data.

To overcome the undesired distortions of the arterial perfusion wave a unique processing algorithm was developed that includes the following steps.
Detection of the cardiac cycles. The detection of the cardiac cycles is usually done by utilizing the R-waves of the ECG signal. Alternatively, the cardiac cycle can be defined from the local maximum of the IPG signal. Both methods can use matched filter to define the peaks of the ECG of the local peaks of the IPG. The raw data is segmented into multiple complexes, representing the perfusion wave dynamic over one cardiac cycle.
Deletion of irregular cardiac cycles. To eliminate variations in the arterial perfusion that are caused by irregular cardiac contractions (and irregular stroke volumes), arrhythmias and cycles with RR interval that significantly deviates from the mean. These irregular complexes are excluded from the analysis. The mean or histogram of the RR are used to identify the most prevalent RR-interval. RR intervals that are outside the a given (usually 2) standard variation from the mean are excluded.
Exclusion of distorted perfusion cycles. The respiratory and slow swings (autonomous) of the raw signal are decomposed from the raw data. Alternatively, morphological characteristics of the cardiac cycle are used for the selection of the appropriate (less distorted) cardiac/perfusion cycle. The morphological characteristics are based on the analysis of the initial and final time segments of each cycle. Each cycle starts a short interval of ΔT (ΔT was usually 100 ms) before the R wave and ends at the time of the next R wave. Thus, there is an overlap of ΔT between the successive heart cycles. The initial and last segments of ΔT in each cycle are analyzed in details (Note that these segments are shorter than the PTT, and therefore do not include the pulse upstroke). The basic underlying assumption is that a repeatable cycle has the same morphology. Therefore, the two segments should be identical (the end each perfusion wave should be identical to the end of the previous wave). The perfusion complex is considered stable if the two segments were alike in their mean amplitude, slope, peak to peak amplitude, and second derivation. The end of the perfusion wave is relatively very smooth and the second derivation should be close to zero (no need for higher order features).

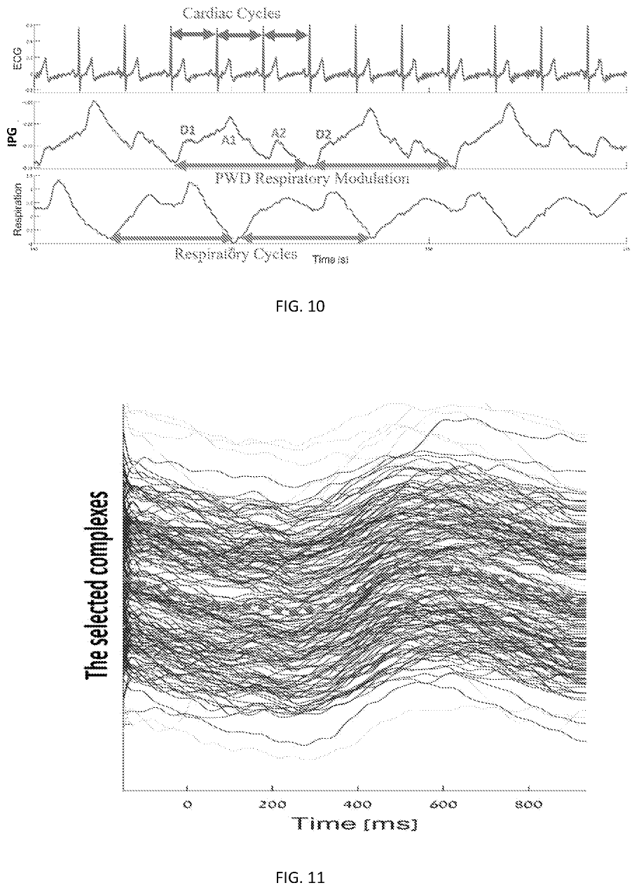

This is a unique algorithm, since simple band-pass filtration and signal decomposition did not work so well. It is important to note that the respiratory waves are not stationary. The respiration is modulated by the voluntary nervous system and is not stationary as the ECG, as depicted in FIG. 10. The respiratory waves change in length, amplitude and shape from one respiratory cycle to the other. There are huge distortions during snoring or speaking. Thus, standard filtrations and decomposition methods that are used for stationary signals are not helpful here, especially when the signal of interest, the perfusion cycle, is usually significantly smaller than the irregular respiratory signal. Thus, instead of trying to correct the distorted perfusion cycles, the distorted cycles are omitted from the analysis.

The acquired data included several minutes of recordings, and hundreds of cardiac cycles (about 350 cycles within 5 minutes). The selection algorithm selects about 50% of the cycles, that were usually acquired during the prolong expiration phase. The selected arterial complexes (FIG. 11) represent the complexes which are not affected by respiratory modulations or random uncontrollable movements.

FIG. 10: The raw impedance plethysmographic signal includes well-defined arterial perfusion waves (e.g. A1, A2) but also significantly distorted perfusion waves (e.g. D1, D2) that are strongly modulated by the respiratory signal. Note also that the respiratory signal is not stationary.

FIG. 11: The selected stable perfusion complexes (bold lines) were averaged to define the ‘characteristic perfusion wave’ (red line). The distorted waves (gray lines) were excluded.

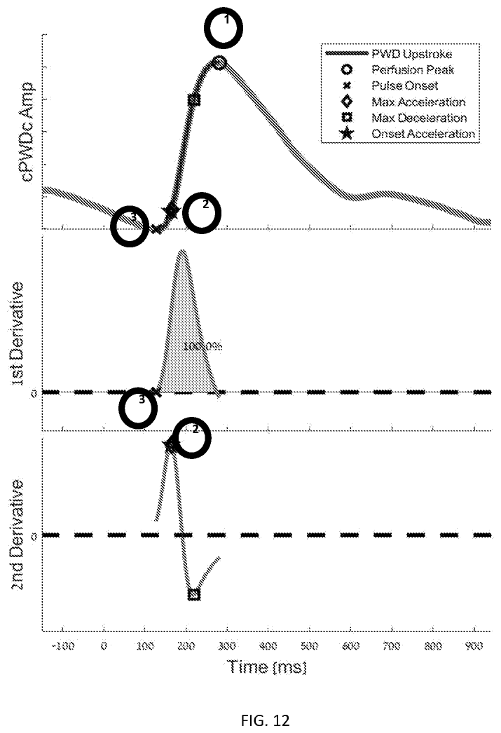

The final step of the analysis performs the feature extraction:
Signal averaging. The selected complexes are averaged to reduce noise and the effects of other random fluctuations, as depicted in FIG. 11, and the averaged ‘characteristic perfusion complex’ is used for further analyses.
Detection of the Peak Perfusion time (PPT). The PPT is the global maximum of the complex (FIG. 12).
Smoothing the signal and calculating the first and second derivative of the signal. In order to detect the next points of interest we need to utilize the first and second derivatives of the signal. This is accomplished by fitting a high degree polynomial (Degree=100). Thereafter, the derivatives are easily attainable by using the fit coefficients and the analytical formula for polynomial derivatives. The first derivative represents the flow. A positive first derivative is an inflow while a negative derivative represents the outflow. The second derivative represents changes in the flow rate.
Detection of the Maximal Acceleration Time (MAT). The MAT is global maximum of the second derivative from the R-wave to the PPT (FIG. 12).
Detecting the perfusion ‘Onset point’. The onset time represents the beginning of the inflow. This point is defined as the earliest local minima with a substantial blood volume inflow following it. The inflow is the area underneath the positive segments of the first derivative (FIG. 12). The onset time is not the global minimum of the signal since the signal may be relatively flat with some noise at the beginning. The onset is the point from which there is substantial filling that is at least a given fraction (about 1%) of the total upstroke.
Identification of the various time segments. The three time points are used for measuring the various time segments, as defined above (FIG. 1): Pulse Transit Time (PTT), Maximum Acceleration Time (MAT), Peak Perfusion Time (PPT), Crest Time (CT), Slow Phase Duration (SPd), and Fast Phase Duration (FPd).

FIG. 12: Identification of the time points for the segmentation of the perfusion upstroke. The Perfusion Peak is defined as the global maximum (point no. 1). The Maximal Acceleration Time (point No. 2) is derived from the second derivative. The first derivative is used to detect the Pulse Onset (Point no. 3), as the point from which there is significant inflow.

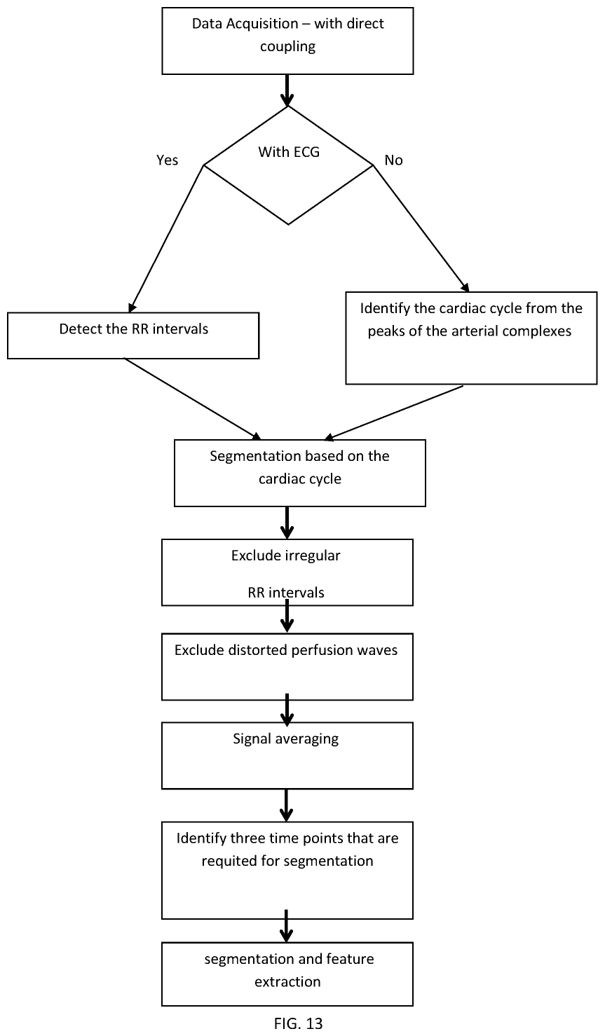

FIG. 13 represents the various steps of the feature extraction algorithm.

The time-frequency domain analysis utilized the wavelet coherence^{14,15} to compare IPG signals from both legs, and produced 4D plots of the correlation, phase-difference, frequency and time.

Statistical Analysis. All average values are presented with the corresponding standard error. The inventors employed the paired t-test to evaluate the statistical significance of the results. P values smaller than 0.05 were considered significant. Signal analysis and statistical analysis were performed using proprietary Matlab (MathWorks Inc., USA) software.

### Results

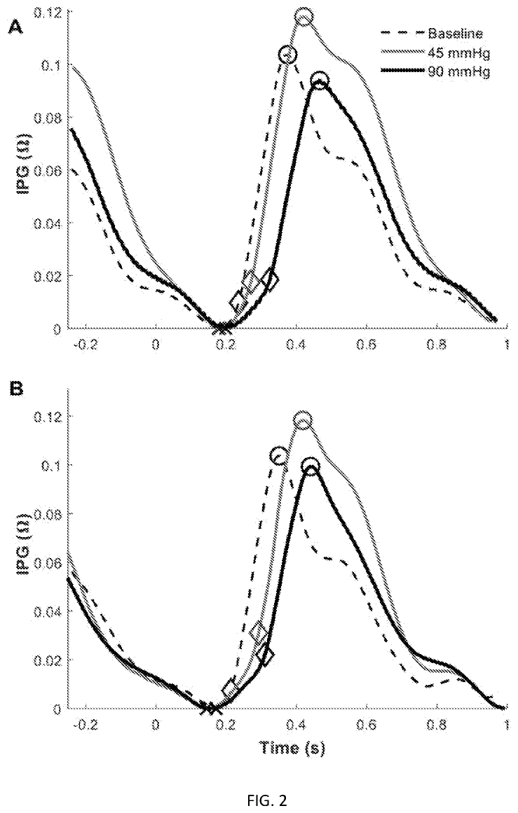

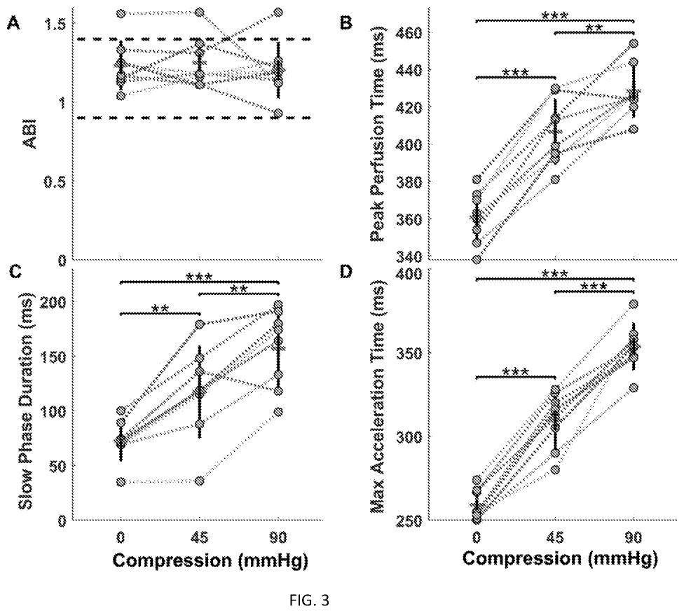

The study enrolled eight healthy female volunteers (27.75±1.3 years old and 58.25±2.4 Kg in weight). FIG. 2 depicts the time course of the IPG signals from the tested leg at baseline and upon 45 and 90 mmHg thigh compressions, in two subjects. Subject A presented a mild decline in the ABI from 1.18 at baseline to 1.11 during 45 mmHg compression and 0.93 at 90 mmHg compression. This subject presented the deepest decline in the ABI in the entire group, but still, the ABI remained above 0.9. Subject B presented smaller variations in the ABI from 1.28 at baseline to 1.11 and 1.19 during 45 and 90 mmHg compressions, respectively.

The IPG in the tested leg and the contralateral leg had almost identical PTT and PPT at baseline. No significant changes were found between the baseline values before 45 and 90 mmHg compressions in all indices. Thus, the inventors present only the 45 mmHg baseline values.

During 45 and 90 mmHg compressions, there were no marked changes in the perfusion transit time (PTT), as shown in FIG. 2. However, the emulated stenoses markedly increased the PPT and the CT in the tested leg. 45 and 90 mmHg compressions increased the PPT in the tested leg from 373 at baseline to 421 ms and 446 ms in subject A, and from 359 ms to 418 ms and 440 ms in subject B. The PPT of the tested leg increased during 90 mmHg compression by 73 ms and 81 ms in subjects A and B, respectively. 45 and 90 mmHg compressions increased the CT of the tested leg from 197 ms at baseline to 240 ms and 268 ms in subject A, and from 182 ms at baseline to 275 ms and 296 ms in subject B. The PPT and CT in the contralateral leg remained constant as at baseline.

The prolongations of the CT and PPT were mainly due to prolongation of the initial slow phase, as depicted in FIG. 2. The slow phase duration (SPd) increased from 58 ms at baseline to 89 ms (+53%) and 126 ms (+117%) during 45 and 90 mmHg compressions, in the tested leg of subject A (FIG. 2A), and from 72 ms at baseline to 149 ms (+107%) and 166 ms (+130%) during 45 and 90 mmHg compressions in subject B (FIG. 2B). Arterial stenosis was also associated with an increase in the relative slow phase-amplitude (SPa). The SPa was only 8.1% (7.1%) of the pulse amplitude at baseline and it increased to 19.7% (22.2%) during 90 mmHg compressions in subject A (B), as depicted in FIG. 2.

The PTT and PPT of the finger-PPG preceded the PTT and PPT of the IPG at baseline (FIG. 1). No significant changes in the timing or shape of the finger-PPG signals were observed throughout the entire experiment.

The PPT of the Hallux-PPG followed the PPT of the IPG (536 ms versus 373 ms in subject A) at baseline. The PPT of the Hallux PPG increased from 536 ms at baseline to 562 ms and 586 ms during 45 mmHg and 90 mmHg compressions (FIG. 1). During 90 mmHg compression, the pulse amplitude of the Hallux-PPG decreased by 50.5% from its baseline value, in subject A.

Table 1 comprises the time-domain results from all subjects (n=8). There was no detectable change in the saturation that was measured from the hallux of the tested leg, during the emulated arterial stenosis, even during 90 mmHg compression, in all the subjects (Table 1):

TABLE 1
The indices from the time domain analyses

| | | | | | | P value | |
|---|---|---|---|---|---|---|---|
| | | | Compression level | | 45 vs | 90 vs | |
| Index | Sensor | Baseline | 45 mmHg | 90 mmHg | Baseline | Baseline | 90 vs 45 |
| SpO₂ (% [symbol] | | 97.0 ± 0.3 | 97.2 ± 0.2 | 97.4 ± 0.4 | 0.593 | 0.348 | 0.513 |
| AB [symbol] | BP | 1.23 ± 0.05 | 1.22 ± 0.06 | 1.18 ± 0.06 | 0.915 | 0.521 | 0.528 |
| PTT (ms [symbol] | IPG | 187 ± 7 | 192 ± 11 | 196 ± 13 | 0.262 | 0.362 | 0.581 |
| PTT (ms [symbol] | PPG | 201 ± 52 | 190 ± 61 | 189 ± 37 | 0.845 | 0.832 | 0.976 |
| PPT (ms [symbol] | IPG | 361 ± 5 | 407 ± 6 | 429 ± 5 | <0.001 | <0.001 | <0.001 |
| PPT (ms [symbol] | PPG | 540 ± 11 | 573 ± 15 | 584 ± 7 | <0.001 | <0.001 | 0.358 |
| CT (ms [symbol] | IPG | 174 ± 8 | 214 ± 12 | 232 ± 11 | 0.003 | <0.001 | 0.030 |
| CT (ms [symbol] | PPG | 339 ± 43 | 383 ± 50 | 395 ± 36 | 0.391 | 0.302 | 0.793 |
| MAT (ms [symbol] | IPG | 259 ± 3 | 310 ± 6 | 354 ± 5 | <0.001 | <0.001 | <0.001 |
| MAT (ms [symbol] | PPG | 401 ± 7 | 440 ± 10 | 465 ± 5 | <0.001 | <0.001 | 0.009 |
| SPd (ms [symbol] | IPG | 73 ± 7 | 117 ± 13 | 157 ± 13 | <0.001 | <0.001 | 0.030 |
| SPd (ms [symbol] | PPG | 200 ± 46 | 249 ± 54 | 276 ± 36 | 0.348 | 0.181 | 0.573 |
| FPd (ms [symbol] | IPG | 102 ± 5 | 97 ± 5 | 75 ± 5 | 0.137 | 0.009 | 0.027 |
| FPd (ms [symbol] | PPG | 139 ± 5 | 134 ± 6 | 119 ± 4 | 0.026 | 0.003 | 0.040 |
| SPa (% [symbol] | IPG | 16.4 ± 2.4 | 22.6 ± 3.2 | 29.5 ± 3.9 | 0.109 | 0.014 | 0.127 |
| SPa (% [symbol] | PPG | 14.3 ± 0.8 | 18.0 ± 1.8 | 20.5 ± 1.0 | 0.064 | <0.001 | 0.171 |
| PA (mΩ [symbol] | IPG | 114 ± 8 | 125 ± 9 | 102 ± 8 | 0.088 | 0.035 | 0.005 |
| PA (mV [symbol] | PPG | 561 ± 135 | 717 ± 162 | 276 ± 77 | 0.115 | 0.034 | 0.021 |
| CW (ms [symbol] | IPG | 57 ± 3 | 66 ± 3 | 59 ± 3 | 0.06 | 0.751 | 0.198 |
| CW (ms [symbol] | PPG | 63 ± 3 | 63 ± 4 | 56 ± 3 | 0.921 | 0.079 | 0.17 |

SpO₂—O₂ Saturation.
ABI—Ankle Brachial Index. [symbol]
indicates data missing or illegible when filed

There were no statistically significant changes in the ABI even during and sustained 90 mmHg compressions (p=0.578). The mean ABI values were 1.23±0.05, 1.22±0.06 and 1.18±0.06 at baseline, 45, and 90 mmHg compressions, respectively, as shown in FIG. A. All ABI values were above 0.9, the threshold for detection of arterial stenosis.

There was no significant difference in the PTT, PPT, MAT, and SPd between the two legs at baseline and these indices remained unchanged throughout the experiments in the contralateral (control) legs and the finger-PPG.

The PTT also did not present significant changes upon 45 and 90 mmHg compressions, in both the IPG and the PPG signals (Table 1). However, the IPG PPT detected all the arterial stenosis events, in all subjects (FIG. B). Both 45 and 90 mmHg compressions induced overt prolongations of the PPT in the tested leg that were proportional to the compression level. The 45 mmHg compression prolonged the PPT by 45.9±4.3 ms (from 361±5 to 406±6 ms, p<0.001). Compression of 90 mmHg prolonged the PPT by 67.8±6.0 ms (to 429±5 ms, p<0.001). The PPT could distinguish between the 45 and 90 mmHg compression levels (p<0.001).

The PPT of the hallux-PPG significantly prolonged during 45 and 90 mmHg thigh compressions; however, the relative changes in the hallux-PPG were smaller than the changes in the IPG. 90 mmHg compression prolonged the PPT of the IPG by 18.09±1.8%, about 2.25 fold the increase in the PPT of the Hallux-PPG that increased by only 8.4±1.3% (Table 1). Moreover, the hallux-PPT did not distinguish between the two stenosis-levels.

The IPG crest-time (CT) increased upon compression and was significantly longer than baseline CT during 45 and 90 mmHg compressions. Moreover, the IPG CT was able to distinguish between the two compression levels. The increase in the CT of the hallux-PPG was not significant compared to baseline and had higher standard error (Table 1).

The MAT that depicts the transition point in the perfusion wave upstroke from the slow-phase to the fast-phase, was sensitive to the presence and severity of stenoses (FIGS. 2,3, Table 1). The IPG MAT prolonged by 50.4±4,8 ms (19.9±1.9%, p<0.001) during 45 mmHg compression and by 94.5±3.5 ms (36.5±1.4%, p<0.001) during 90 mmHg compression. The MATs of both IPG and PPG were able to distinguish between the two thigh compression levels, but the MAT of the hallux-PPG presented smaller changes relative to the IPG.

The fast-phase of the perfusion upstroke dominated the IPG signal amplitude; however, the duration of the IPG fast-phase (FPd) shortened significantly upon 90 mmHg compression (Table 1). Similarly to the IPG, the hallux-PPG fast-phase also significantly shortened during the applied cuff compressions (Table 1). It is important to note that the prolongation of the IPG slow phase was significantly larger than the shortening of the fast phase, at the two compression levels, leading to the significant prolongation of the CT.

The slow phase duration (SPd) significantly prolonged upon 45 and 90 mmHg compressions relative to the baseline (FIG. C, Table 1). Moreover, the SPd could distinguish between moderate (45 mmHg) and severe (90 mmHg) compression levels. Moreover, the relative prolongation of the SPd exceeded the prolongation of the PPT since, at baseline, the SPd was significantly shorter than the PPT. 90 mmHg compression prolonged the IPG SPd by 122±14% and the PPT by only 18.9±1.8%. During compressions, the amplitude of the slow phase (SPa) increased relative to the total pulse amplitude and was statistically significant during 90 mmHg compression (Table 1).

The crest widths of the IPG and PPG did not change significantly during thigh compressions (Table 1). 90 mmHg compression was associated with a mild drop in the IPG pulse amplitude (−10.7±4.3%) and an overt drop in the hallux-PPG pulse amplitude (−35.6±16.1%).

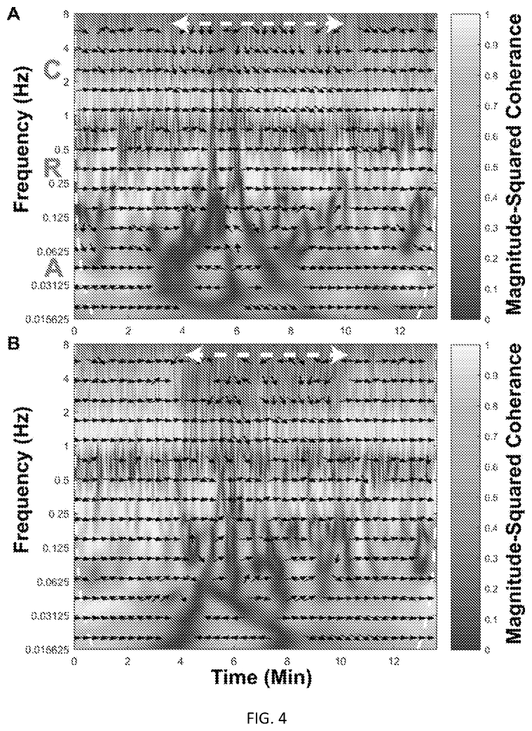

FIG. 4 presents the results of wavelet coherence analysis of IPG data from one subject, during 45 (FIG. A) and 90 mmHg (FIG. B) compressions; The perfusion dynamics is depicted during the three experiment phases: baseline, thigh-compression (4^{th} to 10^{th} minute) and recovery. The correlation (color scale) and the phase differences (black arrows angle) between the IPG-perfusion-waves of both legs are presented at the different frequencies (ordinate) and time points (abscissa).

Three frequency bands are noticeable along the ordinate as horizontal light hue bands, at baseline: The cardiac (denoted as “C”), respiratory (“R”) and the autonomic (“A”) bands. The cardiac band extends from 0.8 to 6 Hz, and most of the energy is between 1 and 2 Hz, as the heart rate of the subject was 82.9±5.9 beats per minute during the entire experiment. The respiratory band resides around 0.25 Hz since the breathing rate was ˜15 breaths per minute. The lowest (autonomic) band was around 0.03 Hz.

The imposed arterial stenosis, by either 45 or 90 mmHg compression, yielded four immediate changes: (1) The correlation in the cardiac-band declined, especially at frequencies above 2 Hz. (2) A phase delay developed between the legs, especially at the cardiac-band. (3) The correlation in the respiratory band attenuated. (4). The correlation in the autonomic band disappeared. These effects were more prominent upon 90 mmHg compression than during 45 mmHg compression (Figure).

The correlation at the 4 to 6Hz band was 0.61±0.06 at baseline and it declined to 0.53±0.08 and 0.36±0.05 during 45 and 90 mmHg compressions, respectively. There was a minor phase difference between the legs at baseline that rose instantaneously upon compression. The phase delay in the cardiac 2 to 4 Hz sub-band grew from 9.0 ±7.2° at baseline to 56.5±9.6° and 69.2±15.4° during 45 and 90 mmHg compressions, respectively.

All changes in correlations and phase faded away upon the release of thigh compression, and the wavelet coherence map during the recovery resembles the map during baseline (Figure).

FIG. 5 compiles the effects of the induced arterial stenosis on the correlation and phase from all subjects. A significant decline in correlation between the tested and the contralateral legs was observed at the cardiac high-frequency sub-band (4 to 6 Hz). The correlation declined from 0.59±0.06 at baseline, to 0.49±0.06 upon 45 mmHg compression (p=0.02) and further to 0.41±0.04 during 90 mmHg compression (p=0.001).

At baseline, the phase difference between legs over the 2-4 Hz band was close to zero (3.3±2.6° (FIG. 5B). The phase difference rose to 34.8±6.6° upon 45 mmHg compression (p=0.001), and further rose to 76.3±1.9° upon 90 mmHg compression (p<0.001). The degree of the phase delay was significantly different in the two stenosis levels (p=0.002).

### Discussion

Our analysis of the perfusion dynamic has provided novel sensitive indices (MAT, SPd, phase delay) that detect the existence of stenosis and quantify its severity. The disclosure highlights the advantages of monitoring the perfusion and of quantifying the perfusion dynamics rather than relating to the single peak pressure point. Stenosis prolongs the peak-perfusion time (PPT) in both the longitudinal IPG and the hallux (microcirculation) PPG. The stenosis prolongs the maximum acceleration time (MAT) without affecting the pulse-transit time (PTT) and the ABI. The disclosure reveals that the perfusion wave upstroke consists of two distinct phases, an initial slow-phase, and a fast-phase. The prolongations of the MAT and PPT are attributed to the prolongation of the low-phase. Arterial stenosis decreases the correlation and generates overt phase delay between the perfusion waves from both legs, in the wavelet coherence maps, at the cardiac frequency band.

### The Slow Phase

Stenosis yielded significant changes in the longitudinal IPG and hallux PPG perfusion waveforms. A prolongation of either the PPT and MAT of the IPG signal enabled detection of stenosis in all the subjects, even during mild compression (45 mmHg). Moreover, the prolongations of the PPT and MAT were proportional to the severity of stenosis and distinguished between the stenosis levels. The PPT is the sum of the PTT and CT, but there were no significant changes in the PTT. Thus, the PPT prolongation can be ascribed to the prolongation of the CT. CT prolongation was also suggested as an index for PAD by others¹¹⁻¹³.

The inventors have divided the CT period at the ‘maximum acceleration point’ into two phases: the initial slow-phase and the fast-phase. The fast-phase represents the overt burst of the perfusion wave. Arterial stenosis prolonged the slow-phase duration (SPd) in both the longitudinal IPG and hallux-PPG, but was statistically significant only in the IPG, during both 45 and 90 mmHg compression. The SPd is, by definition, the difference between the MAT and PTT. There was significant prolongation in the MAT during stenosis, without significant changes in the PTT, in both the longitudinal IPG and hallux-PPG. These trends yielded consistent SPd prolongation in the longitudinal IPG. However, the standard error of the of the hallux-PPG ‘onset time’ (PTT) is larger than the error of the IPG and the prolongation of the MAT is smaller in the hallux-PPG than in the IPG. Thus, the larger variance in the PPG ‘onset-time’ diminished the statistical significance of the observed changes in the SPd of the hallux-PPG. It is important to note that the relative prolongation of the SPd (122±14%) during 90 mmHg compression was significantly larger than in the CT (33.2±3.9%), MAT (36.5±1.4%) and PPT (18.9±1.8%).

The significant prolongations of the CT and PPT are dominated by the prolongation of the MAT and the SPd. The CT is the sum of the SPd and FPd. There were no significant changes in the IPG FPd during 45 mmHg compression, but there was a statistically significant shortening of IPG FPd during 90 mmHg compression and in the hallux-PPG FPd during both 45 and 90 mmHg compressions. However, shortening of the FPd cannot explain prolongation of the CT and PPT. Only the SPd prolongation explains the prolongation of CT, MAT, and PPT. This effect of stenosis on the slow-phase of the perfusion dynamics was not described before. The MAT and SPd are novel indices of arterial stenosis.

### Longitudinal IPG Versus PPG

Many studies have presented the utility of the PPG in detecting PAD^{9,16}. A study that compared the PPG with ABI and angiography suggested the PPG had higher sensitivity than ABI (81.6% versus 69.3%) but lower specificity (77.4% versus 96.8%)⁹. Previous studies have described that the PPG signal is dumped, the dichotic notch is smoothened and the entire PPG signal is diminished in proportion to the severity of the PAD^{9,16}. These changes relate to the PPG amplitude and to shape of the PPG signal following the peak. In contrast, herein the inventors describe significant changes in the perfusion upstroke. Interestingly, the second derivative of the perfusion signal that was used here to define the ‘maximum acceleration point’, was denoted as the ‘a’ wave in previous studies that have analyzed the PPG to assess the severity of vascular aging and arterial stiffness^{12,17,18}. However, they did not use the ‘maximal acceleration point’ to divide the perfusion upstroke into the slow and fast phases and did not describe the effects of stenosis on MAT and the slow-phase duration, as herein.

The longitudinal IPG was superior to PPG in detecting stenosis based on measurements of the PPT, MAT, CT, and SPd. The PPT and MAT were prolonged in both the IPG and PPG, but the nominal (ms) and proportional (%) prolongations in the IPG surpassed those in the PPG. The CT and SPd were statistically significant only with the IPG. The longitudinal IPG relates to changes in the blood volume in the large vessels, while the hallux PPG relates to changes in the microcirculation. The changes in the microcirculation can be also assessed by utilizing transversal IPG with high frequency.

### Novel Indices in the Time Frequency Domain

The emulated arterial stenosis caused significant changes in the shape and phase of the IPG signals that can be quantified by the wavelet coherence analysis. The normal (baseline) IPG signal consists of three frequency bands; cardiac (from 0.8 to 6 Hz), respiratory (around 0.25 Hz) and autonomic (around 0.03 Hz) bands. The respiratory-band reflects the effects of breathing on the venous return, heart rate and cardiac output. The autonomic-band is attributed to myogenic and neurogenic modulation of the circulation by the autonomic nervous system¹⁹.

Femoral artery stenosis caused a decline in the correlation and a significant phase delay buildup between the signals from both legs. The phase delay was proportional to the severity of arterial stenosis and was statistically significant already upon mild (45 mmHg) thigh compression.

A plausible explanation to these observations is based on the adaptive response of the periphery to arterial stenosis. Arterial stenosis is compensated by vasodilatation at the microcirculation, to maintain adequate tissue perfusion^{20,21}. An increase in the resistance in the large artery with adaptive vasodilatation at the microcirculation accentuates the low-pass filtering property of the peripheral vascular tree, which attenuates and delays high frequencies, especially in the cardiac band.

### Perfusion Dynamics Indices and the ABI

Significant changes were observed in the perfusion dynamics indices (PPT, MAT, SPd, phase difference) during the emulated stenosis, without significant changes in the ABI. Sheng et al. have compared the CT and ABI with computer tomography angiography and have shown that the CT (foot to peak) of the pulse wave has better sensitivity than the ABI¹¹. They have suggested that prolongation of the pulse wave CT above 21.7% of the heart cycle enables detection of arterial stenosis at high sensitivity (85%) and specificity (86%)¹¹. The CT in the study for the disclosure was 20.9±0.46% of the heart cycle (174±8 ms) at baseline and it grew to 25.3±1.5% (215±13 ms) and 28.3±1.3% (232±11 ms) upon 45 and 90 mmHg compression, respectively. Although, they have used the oscillometric cuff technique while the inventors have employed the IPG, there is a good agreement between the two studies. Both studies highlight the advantage of utilizing indices of wave dynamics.

A review of eight studies reported that the sensitivity of low ABI (<0.9) in identifying significant arterial stenosis (>50%) is low, from 15% to 79%, especially in the elderly and diabetics⁸. A plausible explanation for the low sensitivity of the ABI is the existence of arteriosclerosis, especially in the elderly and diabetics^{8,10}. Arterial wall hardening causes an overestimation of the leg blood pressure that masks the real decrease in the pressure due to the stenosis. However, here the inventors observed overt changes in the perfusion dynamic in response to arterial stenosis, without noticeable changes in the ABI, in young volunteers without arteriosclerosis. Thus, an additional explanation for the low sensitivity of the ABI relates to Bernoulli's principle²². A significant arterial narrowing with smooth changes in the arterial diameters is associated with insignificant loss of energy and negligible changes in the pressure gradient across the narrowed segment. In a region of severe stenosis, the blood flow velocity increases (by a factor of 4 in 50% stenosis in diameter), and part of the hydrostatic pressure energy turns into kinetic energy. This kinetic energy converts back to hydrostatic energy distal to the stenotic region. Thus, in absence of significant energy loss, and even in the presence of sever stenosis, the pressure at the stenotic region outlet can be close to the pressure at the inlet, yielding normal ABI. However, the changes in the fluid dynamics yield phase delays and slow propagation as was observed here.

### Arteriosclerosis and Arterial Stenosis

Atherosclerosis causes arteriosclerosis and arterial stenosis. Arterial wall stiffening (arteriosclerosis) increases the pressure pulse-wave velocity. The pulse-wave, velocity has a strong predictive value in assessing the risk of cardiovascular events^{23,24}. While the PTT is inversely proportional to the pulse-wave velocity and relates to arteriosclerosis, the novel suggested indices as the PPT, MAT, SPd, and phase-delay are sensitive to the existence of arterial stenosis. The novel analysis suggests that monitoring the perfusion dynamic indices enables to detect arterial stenosis irrespectively of the degree of arteriosclerosis, unlike the ABI¹⁰. This important premise, the ability to differentiate between arteriosclerosis and arterial stenosis, merits further investigation in PAD patients.

### The SPd and the Fractional Flow Reserve

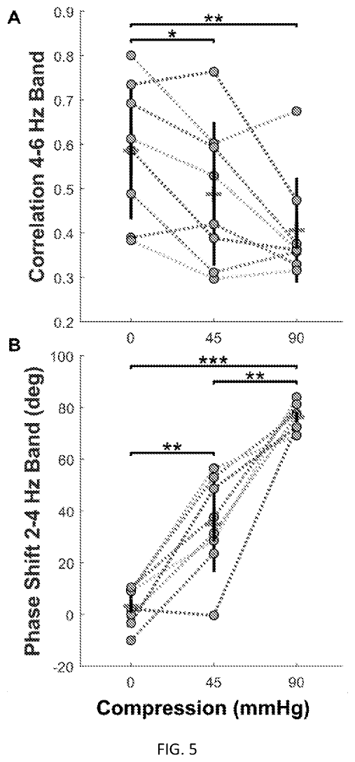

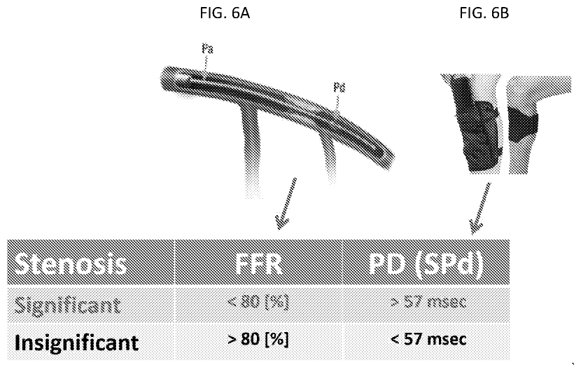

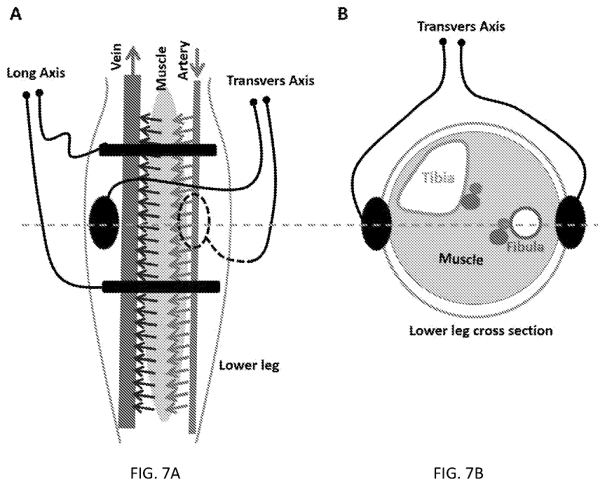

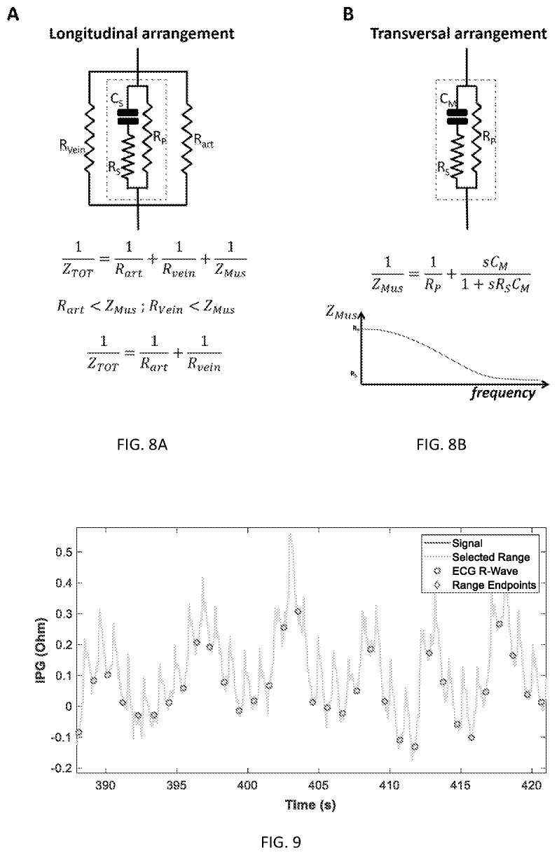

The fractional flow reserve (FFR) is a well-known and acceptable index for quantifying the severity of arterial stenosis and the need for revascularization. The FFR is defines and the ratio of the pressure after the stenosis to the pressure before the stenosis, as depicted in FIGS. 6A. A stenosis that yields FFR smaller than 80% requires revascularization, while revascularization of a stenosis that produces FFR larger than 80% did not improve the outcomes but increases the complication rate. The major limitation of the FFR is that it requires invasive measurement during angiography. The suggested noninvasive technology (FIG. 76B), and the novel indices as the SPd provide similar data. The SPd is an index for flow reserve that directly assesses the changes in the flow dynamics in the presence of stenosis. The SPd can be denoted also as the ‘Perfusion deficit’ in analogue to the ‘fractional flow reserve’ (FFR). Under normal condition the measure ‘SPd’ is short and the interpretation is that there is practically no ‘perfusion deficit’ that require intervention. The longer the SPd the larger is the ‘perfusion deficit’. Severe stenosis is associate with smaller inflow, larger vasodilatation of the microcirculation and consequently the SPd will be prolonged. Narrower stenosis and larger vasodilation will also be associated with smaller FFR (lower distal pressure). Small stenosis with FFR larger than 80% will be associated with larger inflow and smaller adaptive vasodilatation and consequently shorter SPd. Thus the SPd is an index of the ‘perfusion deficit’ (‘PD’ in the table of figures) and is a noninvasive surrogate to the FFR. While the FFR may prevent revascularization when there is no clear indication, the suggested technology can indicate whether there is a need for invasive angiography at all.

Although the suggested technology does not locate the site of stenosis, this is a minor limitation. The main problem is to define whether there is a need for invasive vascular angiography. If there is a need for angiography, it will precisely detect the stenotic site. Thus the technology can prevent unnecessary interventions and improve the screening of patient that can benefit from revascularization.

### Study Limitations

IPG measurements are more cumbersome than PPG or ABI measurements²⁵. However, the study suggests that IPG is more sensitive than the ABI and PPG in the diagnosis of stenosis and differentiating stenosis from arteriosclerosis.

The current study of the invention tested the feasibility of detecting above-knee arterial stenosis by compressing the femoral arteries. The method can be extended to monitor lower arterial stenosis. It can be employed for improving patient followup after above-knee revascularizations.

Arterial stenosis was emulated by inflating a blood-pressure measurement cuff around the thigh that compressed the femoral arteries and veins, and the IPG signal might be affected by the development of venous stenosis. However, the venous drainage does not affect the suggested indices since the venous drainage is modulated by the slow breathing rate (˜0.25 Hz), while the suggested novel indices are derived from the faster cardiac signals (1-6 Hz). Moreover, the indices in the time domain (PPT, MAT, SPd) are derived by averaging the signals from many cardiac cycles and over many breathing cycles, which eliminates the respiratory dependent changes in the venous drainage.

The ABI was measured by the oscillometric technique. Automated oscillometric ABI measurement had been suggested owing to its ease of use²⁶. Oscillometric ABI accuracy had been criticized owing to its limitations in patients with severe arteriosclerosis²⁷. However, in young, healthy subjects with compliant arteries, like the volunteers, there should be no differences between oscillometric and Doppler measurements.

### Conclusion

The disclosure suggests a gamut of novel indices based on the analysis of the perfusion dynamics, as the maximal acceleration time (MAT) and slow-phase duration (SPd), in the time-domain, and the correlation and phase-delay in the time-frequency domain. These indices can detect stenosis in the absence of significant changes in ABI or PTT.

The disclosure divides the perfusion upstroke into the slow and fast phases. A novel finding is that the prolongation in the PPT, MAT, and CT is due to prolongation of the initial slow phase of the perfusion upstroke.

The main advantages of the disclosed technology are:
1. It improves the sensitivity of arterial stenosis detection. Therefore, the technology can be used for screening and for and follow-up after revascularization.
2. It quantifies and assesses the stenosis severity and therefore can improve the diagnosis.
3. It enables to differentiate between general arteriosclerosis and development of focal stenosis. It measures the PTT and quantifies the severity of arteriosclerosis and quantifies the SPd to assess the severity of arterial stenosis, and can differentiate between the two lesions. Therefore, it can detect stenosis irrespectively of the degree of arteriosclerosis and can significantly improve the diagnosis.
4. It can assist in the localization of the arterial stenosis. Although it does not detect the precise location, it identifies whether stenosis exists above the measurement site. Therefore, measurement at level of the calf identifies patients that suffer from large artery stenoses above the level of the knee. Based on the current knowledge and practice, patients that suffer from above-knee stenosis can benefit from revascularization.

### REFERENCES

1. Hirsch A T, Duval S. The global pandemic of peripheral artery disease. The Lancet. 2013;382(9901):1312-1314.

2. Fowkes F G R, Aboyans V, Fowkes F J I, McDermott M M, Sampson U K A, Criqui M H. Peripheral artery disease: epidemiology and global perspectives. Nature Reviews. Cardiology. 2017;14(3):156-170.

3. Shah A D, Langenberg C, Rapsomaniki E, Denaxas S, Pujades-Rodriguez M, Gale C P, Deanfield J, Smeeth L, Timmis A, Hemingway H. Type 2 diabetes and incidence of cardiovascular diseases: a cohort study in 1.9 million people. The Lancet Diabetes & Endocrinology. 2015;3(2): 105-113.

4. Ankle Brachial Index Collaboration. Ankle Brachial Index Combined with Framingham Risk Score to Predict Cardiovascular Events and Mortality: A Meta-analysis. JAMA: the journal of the American Medical Association. 2008;300(2): 197-208.

5. Lee J -Y, Lee S -W, Lee W S, Han S, Park Y K, Kwon C H, Jang J Y, Cho Y -R, Park G -M, Ahn J -M, Kim W -J, Park D -W, Kang S -J, Kim Y -H, Lee C W, et al. Prevalence and Clinical Implications of Newly Revealed, Asymptomatic Abnormal Ankle-Brachial Index in Patients With Significant Coronary Artery Disease. JACC: Cardiovascular Interventions. 2013;6(12): 1303-1313.

6. Gerhard-Herman M D, Gornik H L, Barrett C, Barshes N R, Corriere M A, Drachman D E, Fleisher L A, Fowkes F G R, Hamburg N M, Kinlay S, Lookstein R, Misra S, Mureebe L, Olin J W, Patel R A G, et al. 2016 AHA/ACC Guideline on the Management of Patients With Lower Extremity Peripheral Artery Disease: Executive Summary: A Report of the American College of Cardiology/American Heart Association Task Force on Clinical Practice Guidelines. Circulation. 2017;135(12):e686—e725.

7. Hirsch A T, Criqui M H, Treat-Jacobson D, Regensteiner J G, Creager M A, Olin J W, Krook S H, Hunninghake D B, Comerota A J, Walsh M E, McDermott M M, Hiatt W R. Peripheral Arterial Disease Detection, Awareness, and Treatment in Primary Care. JAMA. 2001;286(11):1317-1324.

8. Dachun Xu null, Jue Li null, Liling Zou null, Yawei Xu null, Dayi Hu null, Pagoto S L, Yunsheng Ma null. Sensitivity and specificity of the ankle-brachial index to diagnose peripheral artery disease: a structured review. Vascular Medicine (London, England). 2010;15(5):361-369.

9. Ro D H, Moon H J, Kim J H, Lee K M, Kim S J, Lee D Y. Photoplethysmography and continuous-wave Doppler ultrasound as a complementary test to ankle-brachial index in detection of stenotic peripheral arterial disease. Angiology.

2013;64(4):314-320.

10. Wikström J, Hansen T, Johansson L, Lind L, Ahlström H. Ankle Brachial Index <0.9 Underestimates the Prevalence of Peripheral Artery Occlusive Disease Assessed with Whole-Body Magnetic Resonance Angiography in the Elderly. Acta Radiologica. 2008;49(2): 143-149.

11. Sheng Chang-Sheng, Li Yan, Huang Qi-Fang, Kang Yuan-Yuan, Li Fei-Ka, Wang Ji-Guang. Pulse Waves in the Lower Extremities as a Diagnostic Tool of Peripheral Arterial Disease and Predictor of Mortality in Elderly Chinese. Hypertension. 2016;67(3):527-534.

12. Wu H -T, Liu C -C, Lin P -H, Chung H -M, Liu M -C, Yip H -K, Liu A -B, Sun C -K. Novel application of parameters in waveform contour analysis for assessing arterial stiffness in aged and atherosclerotic subjects. Atherosclerosis. 2010;213(1):173-177.

13. Maśanauskiené E, Sadauskas S, Naudžiūnas A, Unikauskas A, Stankevičius E. Impedance plethysmography as an alternative method for the diagnosis of peripheral arterial disease. Medicina. 2014;50(6):334-339.

14. Tian F, Tarumi T, Liu H, Zhang R, Chalak L. Wavelet coherence analysis of dynamic cerebral autoregulation in neonatal hypoxic—ischemic encephalopathy. NeuroImage: Clinical. 2016;11:124-132.

15. Tgavalekos K, Pham T, Krishnamurthy N, Sassaroli A, Fantini S. Frequency-resolved analysis of coherent oscillations of local cerebral blood volume, measured with near-infrared spectroscopy, and systemic arterial pressure in healthy human subjects. PLoS ONE. 2019;14(2).

16. Allen J. Photoplethysmography and its application in clinical physiological measurement. Physiological Measurement. 2007;28(3):R1-R39.

17. Otsuka T, Kawada T, Katsumata M, Ibuki C. Utility of second derivative of the finger photoplethysmogram for the estimation of the risk of coronary heart disease in the general population. Circulation Journal: Official Journal of the Japanese Circulation Society. 2006;70(3):304-310.

18. Takazawa K, Tanaka N, Fujita M, Matsuoka O, Saiki T, Aikawa M, Tamura S, Ibukiyama C. Assessment of vasoactive agents and vascular aging by the second derivative of photoplethysmogram waveform. Hypertension (Dallas, Tex.: 1979). 1998;32(2):365-370.

19. Tankanag A V, Grinevich A A, Kirilina T V, Krasnikov G V, Piskunova G M, Chemeris N K. Wavelet phase coherence analysis of the skin blood flow oscillations in human. Microvascular Research. 2014;95:53-59.

20. Johnson P C. Autoregulation of blood flow. Circulation Research. 1986;59(5):483-495.

21. Spaan Jos, Kolyva Christina, van den Wijngaard Jeroen, ter Wee Rene, van Horssen Pepijn, Piek Jan, Siebes Maria. Coronary structure and perfusion in health and disease. Philosophical Transactions of the Royal Society A: Mathematical, Physical and Engineering Sciences. 2008;366(1878):3137-3153.

22. Fox R W, McDonald A T. Introduction to Fluid Mechanics. 5 edition. New York: Wiley; 1998.

23. Niiranen T J, Kalesan B, Larson M G, Hamburg N M, Benjamin E J, Mitchell G F, Vasan R S. Aortic-Brachial Arterial Stiffness Gradient and Cardiovascular Risk in the Community: The Framingham Heart Study. Hypertension (Dallas, Tex.: 1979). 2017;69(6):1022-1028.

24. Ohkuma T, Ninomiya T, Tomiyama H, Kario K, Hoshide S, Kita Y, Inoguchi T, Maeda Y, Kohara K, Tabara Y, Nakamura M, Ohkubo T, Watada H, Munakata M, Ohishi M, et al. Brachial-Ankle Pulse Wave Velocity and the Risk Prediction of Cardiovascular Disease: An Individual Participant Data Meta-Analysis. Hypertension (Dallas, Tex.: 1979). 2017;69(6):1045-1052.

25. Shabani Varaki E, Gargiulo G D, Penkala S, Breen P P. Peripheral vascular disease assessment in the lower limb: a review of current and emerging non-invasive diagnostic methods. BioMedical Engineering OnLine. 2018;17:61.

26. Beckman J A, Higgins C O, Gerhard-Herman M. Automated Oscillometric Determination of the Ankle-Brachial Index Provides Accuracy Necessary for Office Practice. Hypertension. 2006;47(1):35-38.

27. Aboyans V, Criqui M H, Abraham P, Allison M A, Creager M A, Diehm C, Fowkes F G R, Hiatt W R, Jonsson B, Lacroix P, Marin B, McDermott M M, Norgren L, Pande R L, Preux P -M, et al. Measurement and Interpretation of the Ankle-Brachial Index: A Scientific Statement From the American Heart Association. Circulation. 2012;126(24):2890-2909.

What is claimed is:

1. A method comprising:
measuring a perfusion wave upstroke in a perfusion wave associated with leg perfusion dynamics, said perfusion wave upstroke comprising two phases, an initial slow phase and a fast-rising phase, and using prolongation of a duration of said initial slow phase, called a slow phase duration (SPd), to detect a presence of arterial stenosis and to assess stenosis severity.

2. The method according to claim 1, wherein the prolongation of the SPd is proportional to the stenosis severity.

3. The method according to claim 1, wherein the measuring of said perfusion wave upstroke comprises signal processing and segmentation of measured signals into multiple cardiac cycles.

4. The method according to claim 1, wherein the measuring of said perfusion wave upstroke comprises signal processing for removing heart cycle with irregular RR interval.

5. The method according to claim 1, wherein the measuring of said perfusion wave upstroke comprises signal processing for removing heart cycles with distorted perfusion waves.

6. The method according to claim 1, wherein the measuring of said perfusion wave upstroke comprises signal averaging of signals after including signals with similar RR intervals that are not defined as distorted signals.

7. The method according to claim 1, wherein the measuring comprises identification of:
a peak perfusion time point defined as global maximum of the perfusion wave;
a maximal acceleration time point, defined as maximum of a second derivative of the perfusion wave, after smoothing by filtration or high order polynomial curve fitting;
and wherein the method determines the perfusion onset time and differentiates it from the fast perfusion upstroke, wherein the fast perfusion upstroke is the time segment from the MAT to time point of peak perfusion, and the perfusion onset time is the time point from which there is non-negligible filling.

8. The method according to claim 1, further comprising:
measuring pulse transit time (PTT), defined as the time from an ECG R-wave to perfusion onset in the perfusion wave,
measuring peak-perfusion time (PPT), defined as the time from R-wave to peak perfusion point in the perfusion wave,
measuring maximum acceleration time (MAT), defined as the time from R-wave to a maximum acceleration point in the perfusion wave;

9. The method according to claim 8, further comprising using measurements of the PTT, the MAT and the SPd to differentiate between widespread arteriosclerosis and focal arterial stenosis, wherein arteriosclerosis is determined by the pulse wave velocity and the PTT, wherein focal stenosis prolongs the MAT and the SPd.

10. The method according to claim 1, wherein the prolongation of the SPd causes prolongation in a peak-perfusion time (PPT), defined as the time from R-wave to peak perfusion point in the perfusion wave.

11. The method according to claim 1, wherein the prolongation of the SPd causes prolongation in maximum acceleration time (MAT), defined as time from R-wave to a maximum acceleration point in the perfusion wave, the maximum acceleration point being a maximum of a second derivative of the perfusion wave.

12. The method according to claim 1, wherein the prolongation of the SPd causes prolongation in crest-time (CT), defined as the time from perfusion onset to peak perfusion point in the perfusion wave.

13. The method according to claim 1, comprising using the prolongation of the SPd to detect restenosis after revascularization.

14. The method according to claim 1, comprising using the prolongation of the SPd to detect stenosis irrespective of any degree of arteriosclerosis.

15. The method according to claim 1, wherein said measuring comprises measuring signals from electrodes arranged one below another along a longitudinal axis of the leg, and wherein measuring longitudinal impedance plethysmography at a frequency below 100 KHz monitors changes in arterial and venous systems.

16. The method according to claim 1, wherein said measuring comprises measuring signals from electrodes arranged transversely around a circumference of the leg, and wherein transverse impedance plethysmography at a frequency above 100 kHz monitors changes in tissue impedance.
